(19) United States
(12) Patent Application Publication
(10) Pub. No.: US 2009/0281295 A1
(43) Pub. Date: Nov. 12, 2009

(54) CRYSTALLIZATION OF SUCRALOSE FROM SUCRALOSE-CONTAINING FEED STREAMS

(75) Inventors: Edward Micinski, Martinez, GA (US); James Edwin Wiley, JR., Daphne, AL (US); Duane A. Leinhos, Satsuma, AL (US); Mark R. Huber, Singapore (SG)

Correspondence Address: Ratner Prestia, P.O. Box 980, Valley Forge, PA, 19482, US

(73) Assignee: Tate & Lyle Technology Limited, London (GB)

(21) Appl. No.: 12/417,153

(22) Filed: Apr. 2, 2009

Related U.S. Application Data
(60) Provisional application No. 61/042,107, filed on Apr. 3, 2008.

(51) Int. Cl. C07H 17/04

(52) U.S. Cl. 536/18.4

(57) ABSTRACT

A process for the purification of sucralose in sucralose-containing feed streams is disclosed. The process includes multiple liquid-liquid extraction steps; optionally, a concentration step; multiple crystallization steps; optionally, a chromatography step; and various recycle steps.

### CROSS-REFERENCE TO RELATED APPLICATION

This application claims priority from U.S. Provisional Application No. 61/042,107, filed Apr. 3, 2008, which is incorporated herein by reference in its entirety for all purposes.

### FIELD OF THE INVENTION

This invention relates to sucralose and to methods for its preparation. In particular, this invention relates a process for the purification and recovery of sucralose from aqueous sucralose-containing feed streams.

### BACKGROUND OF THE INVENTION

Sucralose (4,1′,6′-trichloro-4,1′,6′-trideoxygalactosucrose), a high-intensity sweetener that can be used in many food and beverage applications, is a galacto-sucrose having the following structure:

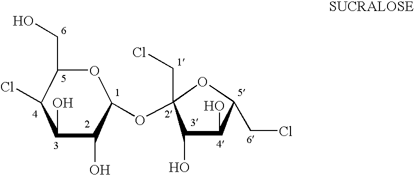

Sucralose is made from sucrose by converting the hydroxyls in the 4, 1′, and 6′ positions to chloro groups. In this process, the stereochemical configuration at the 4 position is inverted.

In one process for making sucralose from sucrose, sucrose is first converted to a sucrose-6-ester, such as sucrose-6-acetate or sucrose-6-benzoate. The sucrose-6-ester is chlorinated by reaction with a chlorination agent and a tertiary amide, and the resulting reaction mixture heated and then quenched with aqueous alkali. The resulting 4,1′,6′-trichloro-4,1′,6′-trideoxygalactosucrose ester (sucralose-6-ester) is converted to sucralose, which is subsequently purified and isolated.

This process typically provides a product that contains varying amounts of other chlorinated sugar compounds in addition to sucralose. During removal of these impurities the loss of sucralose should be minimized, and the purification and isolation process should be economical to operate on a large scale. Although advances have been made in the purification of sucralose, there is a continuing need for processes that remove impurities from sucralose, produce sucralose in high purity, minimize the yield loss in the purification process, and are economical to operate on a large scale.

### SUMMARY OF THE INVENTION

The invention is a process that enhances the yield of sucralose by increasing the purity of the sucralose-containing feed stream fed to the crystallizer. In one embodiment, the process comprises the steps of:

a1) extracting an aqueous feed stream comprising sucralose and chlorinated impurities with a first organic solvent and producing a first organic extract and a first aqueous extract, in which the first organic solvent is immiscible with water, a portion of the sucralose passes into the first organic extract and a portion of the sucralose is retained in the first aqueous extract, and a portion of the chlorinated impurities passes into the first organic extract; and

a2) optionally, extracting the first organic extract with an aqueous solvent and producing a second organic extract and a second aqueous extract, and adding the second aqueous extract to step a1), in which the sucralose preferentially passes into the second aqueous extract, and at least a portion of the chlorinated impurities is retained in the second organic extract;

b) optionally, concentrating the first aqueous extract;

c1) extracting the first aqueous extract with a second organic solvent to give a third aqueous extract and a third organic extract, in which the second organic solvent is immiscible with water, the sucralose preferentially passes into the third organic extract, and a portion of the chlorinated impurities is retained in the third aqueous extract;

c2) optionally, extracting the third organic extract with water to produce a fourth organic extract and a fourth aqueous extract, in which at least a portion of the sucralose is retained in the fourth organic extract, and adding the fourth aqueous extract to at least one of the first aqueous extract and the aqueous feed stream;

d) crystallizing the sucralose from the organic extract produced in step c) and producing a first sucralose product and a first mother liquor, the first mother liquor comprising sucralose and a portion of the chlorinated impurities;

e) carrying out at least one of step e1) and step e2):
e1) adding at least a portion of the first mother liquor to step a2);
e2a) optionally, replacing the second organic solvent of at least a portion of the first mother liquor with water; and
e2b) chromatographing the first mother liquor and producing a purified first mother liquor and a waste stream, in which the sucralose preferentially passes into the purified first mother liquor and at least a portion of the chlorinated impurities preferentially pass into the waste stream; and adding at least a portion of the purified first mother liquor either (i) to at least one of the aqueous feed stream and the first aqueous extract, or (ii) to at least one of step a2), step c2), and step d); and

f) recrystallizing the first sucralose product from an aqueous solvent and producing a second sucralose product and a second mother liquor.

In a first aspect of this embodiment, the first organic solvent and the second organic solvent are the same organic solvent.

In a second aspect of this embodiment, the first organic solvent is ethyl acetate.

In a third aspect of this embodiment, the second organic solvent is ethyl acetate.

In a fourth aspect of this embodiment, step b) is carried out.

In a fifth aspect of this embodiment, the concentration of sucralose in the first aqueous extract is increased by a factor of about 1.15 to 2.5 in step b).

In a sixth aspect of this embodiment, step c2) is carried out.

In a seventh aspect of this embodiment, step c2) is carried out and the fourth aqueous extract is added to the first aqueous extract.

In an eighth aspect of this embodiment, step c2) is carried out and the fourth aqueous extract is added to the aqueous feed stream.

In a ninth aspect of this embodiment, an additional step of extracting the third aqueous extract with the first organic solvent or the second organic solvent and adding the resulting organic extract to at least one of step a2) and step c1) is performed.

In a tenth aspect of this embodiment, step a2) is carried out.

In an eleventh aspect of this embodiment, the chlorinated impurities comprise tetrachloro saccharides, in step a1) greater than 50% of the sucralose and at least 95% of the tetrachloro saccharides in the aqueous feed stream pass into the first organic extract, and in step a2) greater than 90% of the sucralose in the first organic extract passes into the second aqueous extract, and the tetrachloro saccharides are preferentially retained in the second organic extract.

In a twelfth aspect of this embodiment, step e1) is carried out.

In a thirteenth aspect of this embodiment, step e2a) is carried out

In a fourteenth aspect of this embodiment, both step e2a) and step e2b) are carried out.

In a fifteenth aspect of this embodiment, step e1), step e2a) and step e2b) are carried out.

In a sixteenth aspect of this embodiment, step e2b) is carried out and at least a portion of the purified first mother liquor is added to the first aqueous extract.

In a seventeenth aspect of this embodiment, at least a portion of the first mother liquor is added to the aqueous feed stream.

In an eighteenth aspect of this embodiment, step e2b) is carried out and at least a portion of the purified first mother liquor is added to the aqueous feed stream.

In a nineteenth aspect of this embodiment, step e2a) is not carried out.

In a twentieth aspect of this embodiment, step e2b) is carried out and at least a portion of the purified first mother liquor is added to step a2).

In a twenty-first aspect of this embodiment, step e2b) is carried out and at least a portion of the purified first mother liquor is added to step c2).

In a twenty-second aspect of this embodiment, step e2b) is carried out and at least a portion of the purified first mother liquor is added to step d).

In a twenty-third aspect of this embodiment, an additional step of adding at least a portion of the second mother liquor to the first aqueous extract is performed.

In a twenty-fourth aspect of this embodiment, an additional step of adding at least a portion of the second mother liquor to step f) is performed.

In a twenty-fifth aspect of this embodiment, an additional step of replacing the water of at least a portion of the second mother liquor with the second organic solvent and adding a resulting portion of the second mother liquor to step d) is performed.

In a twenty-sixth aspect of this embodiment, the following additional step is performed:

g) recrystallizing the second sucralose product from an aqueous solvent and forming a third sucralose product and a third mother liquor.

In a twenty-seventh aspect of this embodiment, an additional step of adding at least a portion of the third mother liquor (obtained by practice of the twenty-sixth aspect) to at least one of step f) and step g) is performed.

In a twenty-eighth aspect of this embodiment, the following additional step is performed:

h) recrystallizing the third sucralose product (as obtained by practice of the twenty-sixth aspect) from an aqueous solvent and forming a fourth sucralose product and a fourth mother liquor.

In a twenty-ninth aspect of this embodiment, an additional step of adding at least a portion of the fourth mother liquor (as obtained by practice of the twenty-eighth aspect) to at least one of step g) and step h) is performed.

In a thirtieth aspect of this embodiment, an additional step of adding a buffer such as sodium acetate to one of step f), step g), and step h) is performed, in which: the buffer is added to step h) when step g) and step h) are present, the buffer is added to step g) when step g) is present and step h) is absent, and the buffer is added to step f) when both step g) and step h) are absent.

In a thirty-first aspect of this embodiment, the additional step or steps of purifying and isolating the sucralose is or are performed.

In a thirty-second aspect of this embodiment, the first organic solvent and the second organic solvent (which may be the same as or different from each other) are each comprised of a solvent selected from the group consisting of methyl acetate, ethyl acetate, iso-propyl acetate, n-propyl acetate, n-butyl acetate, amyl acetate, methyl ethyl ketone, methyl iso-butyl ketone, methyl iso-amyl ketone, methylene chloride, chloroform, n-butanol, and mixtures thereof; preferably are each comprised of a solvent selected from the group consisting of ethyl acetate, iso-propyl acetate, n-propyl acetate, n-butyl acetate, methyl iso-butyl ketone, n-butanol, and mixtures thereof; and more preferably are each comprised of ethyl acetate.

In a thirty-third aspect of this embodiment, step e2a) is carried out and at least a portion of the first mother liquor is then added to step a1), e.g., combined with the aqueous feed stream.

Two or more of the foregoing aspects of this embodiment of the invention may be practiced in combination with each other, if so desired.

In another embodiment, the process comprises the steps of:

a1) extracting an aqueous feed stream comprising sucralose and chlorinated impurities with a first organic solvent and producing a first organic extract and a first aqueous extract, in which the first organic solvent is immiscible with water, a portion of the sucralose passes into the first organic extract and a portion of the sucralose is retained in the first aqueous extract, and a portion of the chlorinated impurities passes into the first organic extract; and

a2) optionally, extracting the first organic extract with an aqueous solvent and producing a second organic extract and a second aqueous extract, and adding the second aqueous extract to step a1), in which the sucralose preferentially passes into the second aqueous extract, and at least a portion of the chlorinated impurities are retained in the second organic extract;

b) optionally, concentrating the first aqueous extract;

c1) extracting the first aqueous extract with a second organic solvent to give a third aqueous extract and a third organic extract, in which the second organic solvent is immiscible with water, the sucralose preferentially passes into the third organic extract, and a portion of the chlorinated impurities is retained in the third aqueous extract;

c2) optionally, extracting the third organic extract with water to produce a fourth organic extract and a fourth aqueous extract, in which at least a portion of the sucralose is retained in the fourth organic extract, and adding the fourth aqueous extract to at least one of the first aqueous extract and the aqueous feed stream;

d) replacing the second organic solvent of the organic extract produced in step c) with water and producing a fifth aqueous extract;

e) crystallizing the sucralose from the fifth aqueous extract and producing a first sucralose product and a first mother liquor, the first mother liquor comprising sucralose and chlorinated impurities;

f) carrying out at least one of steps f1) and f2):
f1) adding at least a portion of the first mother liquor to step a1);
f2) chromatographing at least a portion of the first mother liquor and producing a purified first mother liquor and a waste stream, in which the sucralose preferentially passes into the purified first mother liquor and chlorinated impurities preferentially pass into the waste stream, and adding at least a portion of the purified first mother liquor to at least one of the first aqueous extract and step e);

g) recrystallizing the first sucralose product from an aqueous solvent and producing a second sucralose product and a second mother liquor.

In a first aspect of this embodiment, the first organic solvent and the second organic solvent are the same organic solvent.

In a second aspect of this embodiment, the first organic solvent is ethyl acetate.

In a third aspect of this embodiment, the second organic solvent is ethyl acetate.

In a fourth aspect of this embodiment, step b) is carried out.

In a fifth aspect of this embodiment, the concentration of sucralose in the first aqueous extract is increased by a factor of about 1.15 to about 2.5 in step b).

In a sixth aspect of this embodiment, step c2) is carried out.

In a seventh aspect of this embodiment, step c2) is carried out and the fourth aqueous extract is added to the first aqueous extract.

In an eighth aspect of this embodiment, step c2) is carried out and the fourth aqueous extract is added to the aqueous feed stream.

In a ninth aspect of this embodiment, an additional step of extracting the third aqueous extract with the first organic solvent or the second organic solvent and adding the resulting organic extract to at least one of step a2) and step c1) is performed.

In a tenth aspect of this embodiment, step a2) is carried out.

In an eleventh aspect of this embodiment, the chlorinated impurities comprise tetrachloro saccharides, in step a1) greater than 50% of the sucralose and at least 95% of the tetrachloro saccharides in the aqueous feed stream pass into the first organic extract, and in step a2) greater than 90% of the sucralose in the first organic extract passes into the second aqueous extract.

In a twelfth aspect of this embodiment, step f1) is carried out.

In a thirteenth aspect of this embodiment, step f2) is carried out.

In a fourteenth aspect of this embodiment, both step f1) and step f2) are carried out.

In a fifteenth aspect of this embodiment, step f2) is carried out and at least a portion of the purified first mother liquor is added to the first aqueous extract.

In a sixteenth aspect of this embodiment, step f2) is carried out and at least a portion of the purified first mother liquor is added to step e).

In a seventeenth aspect of this embodiment, at least a portion of the first mother liquor is added to step e).

In an eighteenth aspect of this embodiment, an additional step of adding at least a portion of the first mother liquor to the aqueous feed stream is performed.

In a nineteenth aspect of this embodiment, an additional step of adding at least a portion of the second mother liquor to step e) is performed.

In a twentieth aspect of this embodiment, an additional step of adding at least a portion of the second mother liquor to step g) is performed.

In a twenty-first aspect of this embodiment, an additional step of adding at least a portion of the second mother liquor to the first aqueous extract is performed.

In a twenty-second aspect of this embodiment, an additional step is performed of:

h) recrystallizing the second sucralose product from an aqueous solvent and forming a third sucralose product and a third mother liquor.

In a twenty-third aspect of this embodiment, an additional step of adding at least a portion of the third mother liquor (obtained by practice of the twenty-second aspect) to at least one of step g) and step h) is performed.

In a twenty-fourth aspect of this embodiment, an additional step is performed of:

i) recrystallizing the third sucralose product (obtained by practice of the twenty-second aspect) from an aqueous solvent and forming a fourth sucralose product and a fourth mother liquor.

In a twenty-fifth aspect of this embodiment, an additional step is performed of adding at least a portion of the fourth mother liquor (obtained by practice of the twenty-fourth aspect) to at least one of step h) and step i).

In a twenty-sixth aspect of this embodiment, an additional step is performed of adding a buffer such as sodium acetate to one of step g), step h), and step i), and, optionally, adding a buffer to at least one of step e1) or step e2), in which: the buffer is added to step i) when step i) and step h) are present, the buffer is added to step h) when step h) is present and step i) is absent, and the buffer is added to step g) when both step h) and step i) are absent.

In a twenty-seventh aspect of this embodiment, an additional step or steps is/are performed of purifying and isolating the sucralose.

In a twenty-eighth aspect of this embodiment, the first organic solvent and the second organic solvent (which may be the same as or different from each other) are each comprised of a solvent selected from the group consisting of methyl acetate, ethyl acetate, iso-propyl acetate, n-propyl acetate, n-butyl acetate, amyl acetate, methyl ethyl ketone, methyl iso-butyl ketone, methyl iso-amyl ketone, methylene chloride, chloroform, n-butanol, and mixtures thereof; preferably are each comprised of a solvent selected from the group consisting of ethyl acetate, iso-propyl acetate, n-propyl acetate, n-butyl acetate, methyl iso-butyl ketone, n-butanol, and mixtures thereof; and more preferably are each comprised of ethyl acetate.

Two or more of the foregoing aspects of this embodiment of the invention may be practiced in combination with each other, if so desired.

### BRIEF DESCRIPTION OF THE DRAWINGS

FIG. 1 is a flow diagram showing a portion of the process of the invention.

FIG. 2 shows the effect of the ratio of organic solvent (“solvent”) to aqueous sucralose-containing feed stream (“feed”) at constant sucralose yield on the purity of the sucralose in the first aqueous extract.

FIG. 3 is a plot showing the effect of carbohydrate concentration on the partition coefficient, K, of sucralose between an organic solvent and water.

FIG. 4 is a plot showing the effect of solvent to feed ratio in extraction EXT2 on the yield of sucralose.

FIG. 5 is a flow diagram showing a portion of one embodiment of the process of the invention.

FIG. 6 is a flow diagram showing a portion of an alternative embodiment of the invention.

FIG. 7 is a plot showing the effect of the purity of the sucralose-containing organic feed stream on sucralose yield.

FIG. 8 is a plot showing the effect of the purity of the sucralose-containing organic feed stream on sucralose yield.

### DETAILED DESCRIPTION OF THE INVENTION

Unless the context indicates otherwise, in the specification and claims, the terms organic solvent, first organic solvent, second organic solvent, tetrachloro saccharide, trichloro saccharide, dichloro saccharide, salt, sucralose-6-ester, carbohydrate, and similar terms also include mixtures of such materials. The term saccharide includes monosaccharide, disaccharides, and polysaccharides. Solvent means a liquid that dissolves another material. An aqueous solvent is one in which water is the primary (greater than 50 vol % of the solvents present) or only solvent. Partition coefficient, K, of a carbohydrate between an organic solvent and water is the concentration of the carbohydrate in the organic phase divided by the concentration of the carbohydrate in the aqueous phase when equal volumes of organic solvent and water are used. Two solvents are immiscible if, in any proportion, they do not form a homogeneous phase. Crystallization includes processes in which a solution is rendered saturated or supersaturated with respect to a dissolved component, and the formation of crystals of this component is achieved. The initiation of crystal formation can be spontaneous, or it can require the addition of seed crystals. Crystallization also describes the situation in which a solid or liquid material is dissolved in a solvent to yield a solution which is then rendered saturated or supersaturated so as to obtain crystals. Also included in the term crystallization are the ancillary processes of washing the crystals with one or more solvents, drying the crystals, and harvesting the final product so obtained. Unless otherwise specified, all percentages are percentages by weight, all temperatures are in degrees Centigrade (degrees Celsius), and all solvent ratios are volume to volume.

A process for the preparation of sucralose from sucrose may involve the following steps. First, the hydroxyl in the 6 position of sucrose is blocked with an ester group, such as acetate or benzoate. Then the hydroxyls in the 4, 1′, and 6′ positions of the resulting sucrose 6-ester are converted to chloro groups, with inversion of the stereochemical configuration at the 4 position. Conversion of the hydroxyls in the 4, 1′, and 6′ positions of the ester to chloro groups with inversion of the stereochemical configuration at the 4 position is disclosed in Walkup, U.S. Pat. No. 4,980,463; Jai, U.S. Pat. Pub. 2006/0205936 A1; and Fry, U.S. Pat. Pub. 2007/0100139 A1; the disclosures of which are all incorporated herein by reference in their entirety for all purposes. Then the ester group in the 6 position of the resulting sucralose-6-ester is removed, and sucralose, the resulting product, purified and isolated. The process, or any of the individual steps thereof, can be either a batch or a continuous process.

### Preparation of Sucralose-Containing Feed Streams

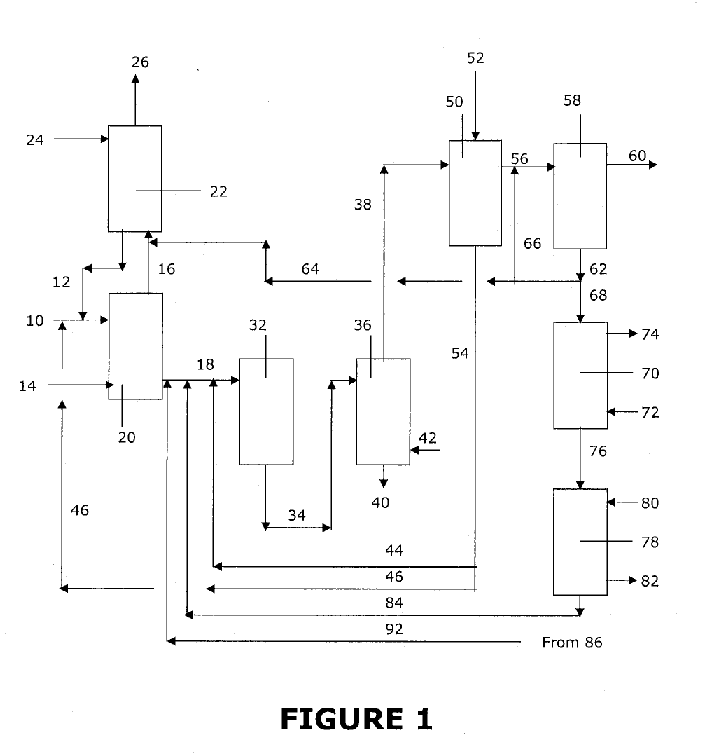

Referring to FIG. 1, following conversion of sucralose-6-ester to sucralose, an aqueous feed stream (10) that comprises sucralose is produced. Aqueous feed stream 10 typically comprises a total of about of 6 wt % to 50 wt %, for example, about 6 wt % to 12 wt %, about 12 wt % to 18 wt %, about 18 wt % to 25 wt %, or about 25 wt % to about 50 wt % of carbohydrates in a stream in which water is the primary or only solvent. Of the carbohydrates present, between 50% and 80% are typically sucralose. The other carbohydrates primarily fall into one of three categories based on the number of chlorine atoms on the molecule: tetrachloro saccharide impurities (tetrachloro saccharides), dichloro saccharide impurities (dichloro saccharides), and trichloro saccharide impurities (trichloro saccharides). The location and extent of chlorination strongly affect the polarity of the resulting saccharide. In general, the tetrachloro saccharides impurities are less polar than sucralose, and the dichloro saccharide impurities are more polar than sucralose. Generally, more polar impurities are more soluble than sucralose in more polar solvents, and less polar impurities are more soluble than sucralose in less polar solvents.

Other materials that can be present in aqueous feed stream 10 include inorganic salts, such as alkali metal chlorides such as sodium chloride, alkaline earth chlorides, and ammonium chloride; and organic salts, primarily alkali metal acetates, such as sodium acetate; dimethyl amine hydrochloride; and alkali metal formates, such as sodium formate. A small amount, typically less than 5,000 ppm, of the polar aprotic solvent used in the chlorination step, typically N,N-dimethyl formamide, can also be present in the feed stream.

Aqueous feed stream 10 and, if present, second aqueous extract 12, discussed below, are combined to produce a combined aqueous stream, which is extracted with a stream of a first organic solvent (14) to produce a first organic extract (16) and a first aqueous extract (18). This extraction step is referred to as step EXT1. Because the less polar compounds are preferentially extracted into first organic extract 16, this extraction removes less polar compounds, which include the tetrachloro saccharides, as well as a portion of the sucralose, from the combined aqueous stream. The extraction can be carried out under conditions in which greater than 50%, greater than 55%, greater than 60%, or greater than 65%, of the sucralose and 95% of the tetrachloro saccharide impurities in the aqueous feed stream are extracted into first organic extract 16. In an alternative embodiment, the extraction can be carried out as disclosed in WO03/076453, namely wherein a majority (i.e., greater than 50%) of the tetrachlorosucrose compounds in the aqueous feed stream 10 are extracted into the first organic extract 16, and a majority (i.e., greater than 50%) of the sucralose is retained in the first aqueous extract 18.

The choice of solvent is determined by the relative solubilities of sucralose and the principal impurities in the organic solvent and in the aqueous feed stream, as well as such other factors as flammability, ease of recycling within the process, environmental concerns, toxicity, and cost. If desired, the organic solvent can be intentionally saturated with water before use in the extraction step. Mixtures of organic solvents can be used. Solvents contemplated for use as the first organic solvent include those that are immiscible with water and in which halogenated sucrose derivatives, such as sucralose, are readily soluble. Also included are solvents that are partially soluble in a first solvent such as water, an aqueous solution, or other solvent in which halogenated sucrose derivatives are readily soluble, but in which the second solvent still forms a separate phase when mixed with the first solvent in proper ratios and under proper conditions. Typical first organic solvents include, but are not limited to, methyl acetate, ethyl acetate, methyl ethyl ketone, methyl iso-butyl ketone, methyl iso-amyl ketone, methylene chloride, chloroform, diethyl ether, methyl t-butyl ether, n-pentane, n-hexane, n-heptane, n-octane, isooctane, 1,1,1-trichloroethane, n-dodecane, white spirit, turpentine, cyclohexane, propyl acetate, butyl acetate, amyl acetate, carbon tetrachloride, xylene, toluene, benzene, trichloroethylene, 2-butoxyethanol acetate (butyl CELLOSOLVE® acetate), ethylene dichloride, butanol, morpholine, and mixtures thereof. The first organic solvent preferably comprises methyl acetate, ethyl acetate, iso-propyl acetate, n-propyl acetate, n-butyl acetate, amyl acetate, methyl ethyl ketone, methyl iso-butyl ketone, methyl iso-amyl ketone, methylene chloride, chloroform, or n-butanol, either as a single solvent, or as a mixed solvent with these solvents, or with other solvents from the first list. The first solvent more preferably comprises ethyl acetate, iso-propyl acetate, n-propyl acetate, n-butyl acetate, methyl iso-butyl ketone, or n-butanol, either as a single solvent, or as a mixed solvent with these solvents, or with other solvents from the first or second list. Ethyl acetate is the most preferred solvent. Diethyl ether, methyl t-butyl ether, n-pentane, n-hexane, n-heptane, n-octane, isooctane, 1,1,1-trichloroethane, n-dodecane, white spirit, turpentine, cyclohexane, carbon tetrachloride, xylene, toluene, benzene, trichloroethylene, 2-butoxyethanol acetate (butyl CELLOSOLVE® acetate), ethylene dichloride, and morpholine are generally not preferred as single solvents, but may be used in mixed solvents as described.

Extraction is carried out in a first liquid extractor (20), which can be any type of liquid-liquid extractor known in the art, for example, a conventional mixer-settler or a bank of conventional mixer-settlers, an Oldshue-Rushton multiple-mixer column, a sieve tray column, a random packed column, a pulsed packed column, a structured (SMVP) packing column, an asymmetric rotating disk extractor (ARD), a KARR® column, a Kuhni extractor, a Treybel extractor, a Scheibel column, a rotating disc contactor (RDC) column, or a centrifugal extractor such as a Podbielniak centrifugal extractor or a Robatel centrifugal extractor. An extractor with five or more theoretical stages of extraction can be used. A first organic solvent 14, for example ethyl acetate which, if desired, has been saturated with water, is fed to the bottom of extractor 20 in proportion to the total amount of feed to the top of extractor 20.

First aqueous extract 18 comprises sucralose as well as some impurities, primarily salts and saccharide impurities that are more polar than sucralose or which have about the same polarity as sucralose. First aqueous extract 18 (together, optionally, with one or more recycled sucralose-containing aqueous streams as described in more detail below, collectively referred to as “feed stream 18”) is used as the feed stream for additional purification steps, described below.

If desired, first organic extract 16 can be sent to a second liquid extractor (22) to recover sucralose from first organic extract 16 while leaving the bulk of the less polar impurities in an organic extract. This extraction step is referred to as step EXT1B. As discussed below, recycle streams from later purification steps can be recycled to the second liquid extractor 22. Second liquid extractor 22 can be any type of liquid-liquid extractor known in the art, examples of which are listed above. An extractor with five or more theoretical stages of extraction can be used. First organic extract 16 is fed into the bottom of liquid extractor 22. A stream (24) of water, which if desired can be saturated with the same organic solvent used in first liquid extractor 20, for example water saturated with ethyl acetate, is fed into the top of extractor 22. The mass ratio of water to first organic extract 16 is typically about 0.8 to about 0.9. An interface between the two phases is maintained in the bottom of second liquid extractor 22 where the aqueous phase, second aqueous extract 12, is collected. Second aqueous extract 12 can be recycled to first liquid extractor 20, typically by being combined with aqueous feed stream 10. Greater than 85%, 90%, 92%/, or 95% of the sucralose present in the first organic phase is extracted into the second aqueous phase by step EXT1B.

The organic extract, second organic extract 26, exits the top of extractor 22. Second organic extract 26 contains less polar impurities, such as the tetrachloro saccharides. It is purged from the process, and the organic solvent recovered for reuse. If the second liquid extraction (step EXT1B) is not present in the process, the first organic extract is purged from the process, and the organic solvent recovered for reuse.

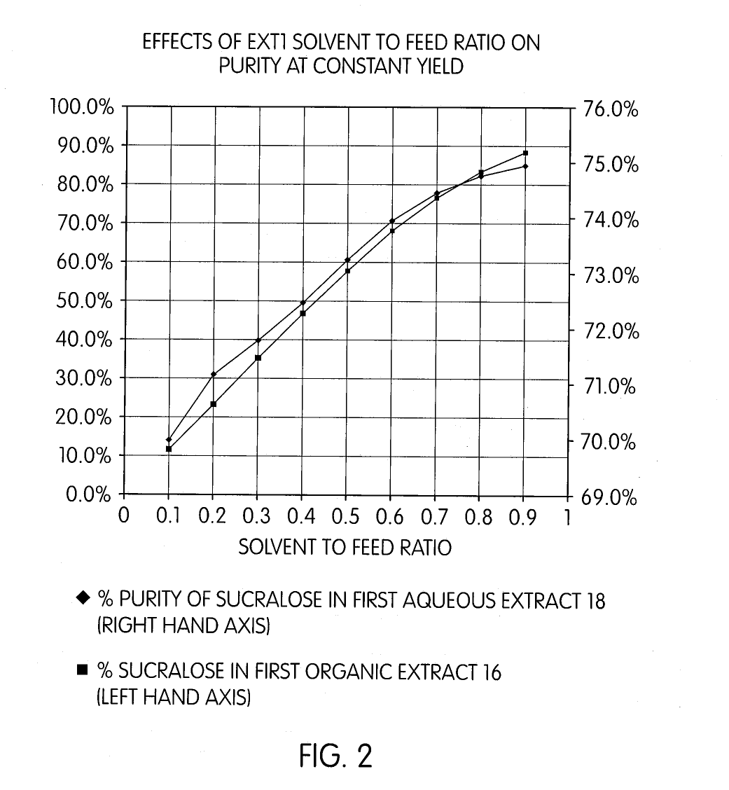

In one aspect of the invention, the mass ratio of first organic solvent 14 to aqueous feed stream 10 in the first extraction step (EXT1) is about 0.4 to about 0.9. Preferably, the mass ratio of first organic solvent 14 to aqueous feed stream 10 in step EXT1 is about 0.6 to about 0.9. FIG. 2 shows the amount of sucralose in first organic extract 16 (left hand axis) and the purity of the sucralose in first aqueous extract 18 (right hand axis) as a function of mass ratio of first organic solvent 14 to aqueous feed stream 10 in the first extraction step (EXT1), calculated at constant sucralose yield. These values are for the process described above, a process in which a sucralose-containing aqueous feed stream is extracted with an organic solvent in a first extraction step, the resulting organic extract back is extracted with water in a second extraction step, and the resulting second aqueous extract is recycled to the first extraction step.

As can be seen from FIG. 2, when the mass ratio of organic solvent 14 to aqueous feed stream 10 in the first extraction step is about 0.4 or greater, about 50% or more of the sucralose is extracted into first organic extract 16. When the ratio is 0.6 or greater, greater than about 65% of the sucralose is extracted into first organic extract 16. Surprisingly, the level of impurities in first aqueous extract 18 is reduced significantly, without yield loss of overall sucralose yield, when higher organic solvent to aqueous feed stream mass ratios are used in the first extraction step.

Referring to FIG. 1, in one aspect of the invention, if desired, feed stream 18 (which comprises the first aqueous extract together, optionally, with one or more other aqueous streams such as, for example, stream 44 of fourth aqueous extract 54, aqueous sucralose-containing stream 84, or stream 92 of second mother liquor 90) can be fed to a concentrator (32), which can be designed for either a batch or continuous operation. Concentrator 32 can be any type of evaporator known in the art, for example, a falling film evaporator, a thin-film evaporator, a wiped film evaporator, a forced circulation evaporator, a bulk evaporator, a Robert evaporator, a Herbert evaporator, a Caddle-type evaporator, or an Oskar evaporator. As described below, other sucralose-containing aqueous streams can be recycled to concentrator 32.

Concentrator 32, if present, increases the concentration of carbohydrates, including the concentration of sucralose, and, if present, the salt present in sucralose-containing aqueous feed stream 18 (which comprises first aqueous extract 18, optionally in combination with one or more recycled sucralose-containing aqueous streams). Concentrator 32 typically increases the concentration of carbohydrates in sucralose-containing aqueous feed stream 18 by a factor of about 1.1 to about 4.0, or about 1.15 to 2.5, or about 1.2 to about 2.0. Sucralose-containing aqueous feed stream 18 entering concentrator 32 can have less than about 18 wt %, less than about 15 wt %, less than about 12 wt %, less than about 10 wt %, less than 9 wt %, or less than 8 wt % total carbohydrates, and more than 3 wt %, or more than 4 wt %, or more than 5 wt %, for example, about 3 wt % to about 18 wt %, 4 wt % to about 16 wt %, about 4 wt % to about 15 wt %, about 4 wt % to about 12 wt %, about 4 wt % to about 10 wt %, about 4 wt % to about 8 wt %, or about 5 wt % to about 8 wt %, total carbohydrate. Sucralose-containing aqueous feed stream 18 may contain up to about 18 wt % or even more of inorganic salts, primarily alkali metal chlorides, such as sodium chloride, and organic salts, primarily alkali metal acetates, such as sodium acetate. Concentrated sucralose-containing aqueous feed stream 36 leaving concentrator 32 can have at least about 10 wt %, at least about 12 wt %, at least about 13 wt %, at least 15 wt %, at least 18 wt %, at least 20 wt %, at least 22 wt %, or at least 25 wt %, and 50 wt % or less, 45 wt %, or 40 wt % or less total carbohydrate; for example, about 10 wt %, about 12 wt %, about 15 wt %, or about 18 wt % to about 25 wt %; about 10 wt %, about 12 wt %, about 15 wt %, or about 18 wt % to about 20 wt %; about 10 wt %, about 12 wt %, or about 15 wt % to about 18 wt %; about 13 wt % to about 17 wt %; about 14 wt % to about 16 wt %; or about 15 wt % to about 16 wt % total carbohydrate. Typically, sucralose comprises about 60% to 85% of the carbohydrates present in concentrated sucralose-containing aqueous feed stream 34.

Feed stream 18 (comprising first aqueous extract 18 together, optionally, with one or more other aqueous streams such as, for example, stream 44 of fourth aqueous extract 54, aqueous sucralose-containing stream 84, or stream 92 of second mother liquor 90), or, if concentrator 32 is present, concentrated aqueous stream 34 is fed to third liquid extractor 36. This extraction step is referred to as step EXT2 and can be either batch or continuous. Third liquid extractor 36 can be any type of liquid-liquid extractor known in the art, examples of which are given above. In this extraction step, sucralose is extracted into a second organic solvent (42) to form a third organic extract (38). Most of the more polar impurities and most of the salts present in the aqueous feed remain in the third aqueous extract (40). Third aqueous extract 40 exits the bottom of third liquid extractor 36 and is purged from the process.

Alternatively, if desired, third aqueous extract 40 can be back extracted with an organic solvent, such as the first or second organic solvent, for example ethyl acetate, before being purged from the process. The recycle stream from this back extraction of third aqueous extract 40 can be combined with stream 42 (the solvent feed to extractor 36) or the recycle stream can be fed to second liquid extractor 22.

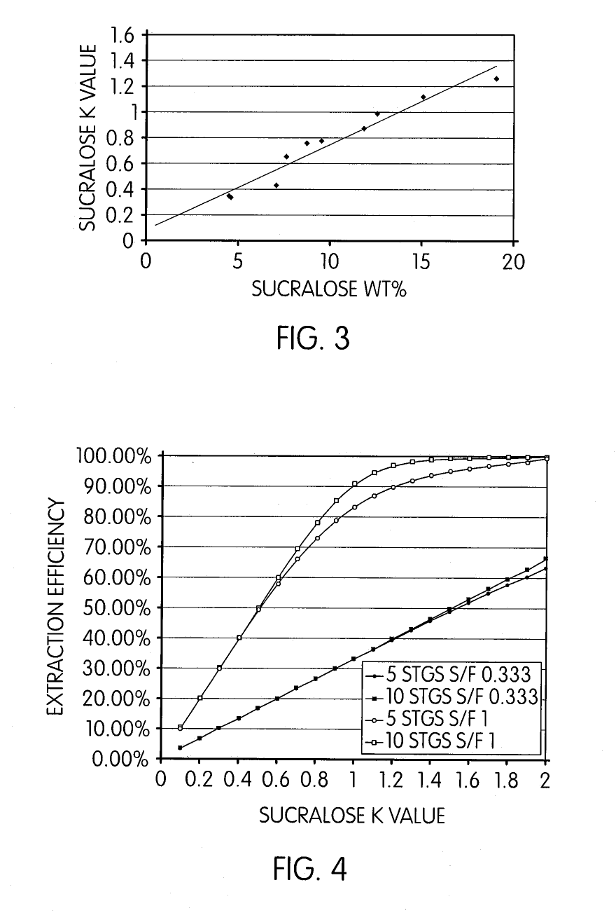

Surprisingly, K, the partition coefficient for sucralose between an organic solvent and water when equal volumes of organic solvent and water are used is dependent on the concentration of carbohydrates. As shown in FIG. 3, when equal volumes of ethyl acetate and water are used, K increases from 0.4, when the initial concentration of sucralose in the aqueous phase is about 5 wt % to about 1.1 when the initial concentration of sucralose in the aqueous phase is about 15 wt % and about 1.2 when the initial concentration of sucralose in the aqueous phase is about 16 wt %. As shown in FIG. 4, when the ratio of organic solvent to water is 1, the extraction efficiency increases from about 40% to about 90% when the K value increases from about 0.4 to about 1.1 and the extraction efficiency increases from about 40% to over 90% when the K value increases from about 0.4 to about 1.2. Therefore, before the sucralose is extracted into an organic solvent, it is advantageous that sucralose-containing aqueous feed stream 18 be concentrated before extraction with the organic solvent. Preferably, the extraction can be carried out under conditions at which the partition coefficient for sucralose between the organic extract and the aqueous extract is at least about 1.0, more preferably at least about 1.1 or at least about 1.2. The partition coefficient is typically in the range of about 1.0 to about 1.6, about 1.1 to about 1.6, about 1.2 to about 1.6, or about 1.25 to 1.6.

Feed stream 18 (comprising first aqueous extract 18 together, optionally, with one or more other aqueous streams such as, for example, stream 44 of fourth aqueous extract 54, aqueous sucralose-containing stream 84, or stream 92 of second mother liquor 90), or, if concentrator 32 is present, concentrated aqueous stream 34 is fed to the top of a third liquid extractor (36) and a stream of second organic solvent 42, for example a stream of ethyl acetate which if desired may be saturated with water, is fed to the bottom of extractor 36. The mass ratio of organic solvent 42 to aqueous feed stream 34 is in the range of about 1.5 to about 4.0, for example about 1.5 to about 2.0, or about 2.0 to about 2.5, or about 2.5 to about 4.0. However, if the number of theoretical stages of extraction in third liquid extractor 36 were increased, the amount of organic solvent 42, and consequently the ratio of organic solvent 42 to aqueous feed stream 34, could be reduced.

Any of the organic solvents used as the first organic solvent can be used as the second organic solvent. However, as a result of this extraction step, sucralose is transferred from an aqueous extract to an organic extract, so, if sucralose is to be crystallized from the organic solvent, it is convenient to use a second organic solvent that can be used as the crystallization solvent for sucralose. It is also convenient for the first organic solvent and the second organic solvent to be the same organic solvent. A preferred second organic solvent is ethyl acetate.

### Crystallization of Sucralose

Sucralose can be isolated by crystallization from a sucralose-containing organic feed stream. However, it has been discovered that the purity of the feed to the crystallization step affects sucralose yield. Lower feed purity produces a lower yield and ultimately lower overall plant yield because a larger amount of sucralose is removed with impurities in the mother liquor.

Referring to FIG. 1, third organic extract 38, which contains sucralose, exits the top of third liquid extractor 36 and, optionally, is fed to fourth liquid extractor 50. This extraction step is referred to as EXT2B and can be either a batch or continuous process. Fourth liquid extractor 50 can be any type of liquid-liquid extractor known in the art, examples of which are listed above. A stream (52) of water, which if desired is saturated with the second organic solvent, for example water saturated with ethyl acetate, is added to extractor 50. Use of a single stage or a multi-stage counter-current liquid-liquid extraction in the back extraction increases the overall yield of the entire isolation and purification process by 1-2%. This step further reduces the salt content of the organic extract and increases the purity of the feed to the crystallization step. Because fourth aqueous extract 54 contains sucralose as well as salts, a stream (44) of fourth aqueous extract 54 can be recycled to concentrator 32, and/or, if concentrator 32 is not present, to extractor 36 for recovery of additional sucralose. Alternatively, or additionally, a stream (46) of fourth aqueous extract 54 can be recycled to aqueous feed stream 10 for recovery of additional sucralose.

Third organic extract 38, or if fourth liquid extractor 50 is present, fourth organic extract 56 is fed to a first crystallizer (58). This step can be either a batch or a continuous process. First crystallizer 58 can be any type of crystallizer known in the art, for example, a Swenson-Walker crystallizer, a mixed tank crystallizer, a fluidized bed crystallizer, a draft tube baffle (DTB) crystallizer, a Krystal continuous crystallizer, a forced circulation evaporative crystallizer, an Oslo type or classified-suspension crystallizer, or an induced circulation crystallizer. In first crystallizer 58, sucralose is separated from a majority of the trichloro saccharides as well as from other impurities. Because the sucralose has been extracted into the second organic solvent, the crystallization solvent is the second organic solvent, for example, ethyl acetate.

Operation of the crystallizer will be determined by, for example, whether the crystallization process is batch or continuous; the type and design of crystallizer chosen; the properties of the crystallization solvent chosen, including, for example, its boiling point, its heat of vaporization, and the solubility of sucralose and the impurities as a function of temperature in the chosen solvent; the concentration of sucralose in the feed to the crystallizer; the purity of the feed to the crystallizer; the nature of the impurities in the feed; mixing requirements of the crystallizer; seeding requirements; and solid-liquid separation requirements; as well as the crystal size, crystallization rate, product yield, and product purity desired. The temperature of the solvent in the crystallizer can be controlled by a number of means. A jacketed vessel or a vessel with one or more internal cooling coils can be used. The solution/slurry in the crystallizer can be pumped through an external heat exchanger. Evaporative cooling can be used for temperature control by altering the pressure in the crystallizer, which, in turn, controls the boiling point of the solvent. Too high a temperature can cause product degradation. If the temperature is too low, there may not be enough heat available to evaporate the solvent. Some of the variables that affect the crystallization are the density or specific gravity of the slurry in the crystallizer, mixing intensity, and crystallization rate. In a batch crystallizer, evaporation of the solvent can be used to concentrate the solution, and the sucralose can be crystallized by cooling the solution. If necessary, crystallization can be induced by, for example, addition of seed crystals. In a continuous crystallizer, such factors as feed rate, slurry density, residence time of the sucralose in the crystallizer, and manner of product removal from the crystallizer need to be considered.

Crystallization produces a first sucralose product (60) and a first mother liquor. First sucralose product 60 can be separated from the first mother liquor by any convenient solid-liquid separation technique known in the art, such as filtration, for example, by pressure filtration, rotating filters, continuous rotary vacuum filters, continuous moving bed filters, or batch filters, or by batch or continuous solid-liquid centrifugation. First sucralose product 60 is further purified by additional processing steps, described below.

Referring to FIG. 1, a stream of first mother liquor 62, which contains sucralose in addition to impurities, exits first crystallizer 58. First mother liquor 62 can be recycled to the process by one or more routes. If second liquid extractor 22 is present, a stream (64) of a portion or all of the first mother liquor can be recycled to second liquid extractor 22. This step produces significant purification of sucralose with very little loss of yield. Overall yields are maintained at a high level, and additional purge streams that would reduce sucralose yield are not required to remove less polar impurities from the process. It is also sometimes advantageous to recycle a small stream (66) of the first mother liquor back to first crystallizer 58.

Alternatively, or additionally, a stream (68) of a portion or all of the first mother liquor can be purified by chromatography. In one aspect of the invention, prior to chromatography, the organic solvent is replaced with water. This can be carried out using a distillation column (70). Stream 68 of first mother liquor is fed to distillation column 70. A stream of water (72) is added and the organic solvent (74) distilled off and recovered for reuse in the process. When the organic solvent is ethyl acetate, the ethyl acetate-water azeotrope can be removed by distillation. Alternatively, the organic solvent can be distilled off or stripped off under reduced pressure, and the resulting solid material, the solids formerly dissolved in stream 68 of first mother liquor, dissolved in water.

The resulting stream (76) of aqueous first mother liquor is fed to a chromatography column (78). Various methods can be used for chromatographic purification of sucralose. For example, two simulated moving bed type units can be run in series. A slow purge is taken from one unit, and a fast purge is taken from the other unit, with concentrating in between. Alternatively, a single sequential simulated moving bed system with three product streams can be used. Chromatographic purification of sucralose is described, for example, in Catani, U.S. Pat. No. 5,977,349, the disclosure of which is incorporated herein by reference in its entirety for all purposes.

If desired, chromatography column 78 can comprise a sacrificial resin bed, or guard bed through which stream 76 is passed prior to being fed to the separation column. The guard bed does not separate sucralose from its impurities; instead, it adsorbs materials that interfere with the separation of sucralose from the impurities. Cation exchange resins that have sulfonate groups attached to a crosslinked polystyrene backbone, such as Purolite C120E and DOWEX® HCRS cation exchange resins, perform well as guard bed resins. Because it is necessary to stop the process in order to regenerate the resin by washing, resulting in lost production time, two guard bed columns can be used. One is prepared for use while the other is in use so that it is unnecessary to interrupt the process to regenerate the guard bed.

The absorbent used for separation preferably has a large surface area for adsorption. This can be obtained, for example, by using a resin in the form of small beads, a macroporous resin with large cavities in the otherwise spherical resin bead, or a gel resin in which the beads can be penetrated by the molecule, thereby allowing the molecule to access the interior of the beaded resin. Resins effective in adsorbing sucralose from an aqueous medium include non-polar, unsubstituted macroporous polymeric resins such as the AMBERLITE® XAD series resins (for example, AMBERLITE® XAD16 resin). Resins effective at separation using small beads include most analytical reverse phase C-18 and non-polar beaded resins such as PRP-1. Also effective are REZEX® RNM cation substituted polystyrene analytical resins. The substituted gel resins of about 40% or less divinylbenzene crosslink are particularly useful as separation resins. The ionic substituent adds sufficient polarity to the polystyrene/divinylbenzene backbone to allow sucralose and its impurities to elute at different rates in an aqueous medium. Preferred resins are those substituted with a sulfonate group that can exchange cations. Particularly preferred are MITSUBISHI® UBK510L, DOWEX® 50×4, and FINEX® CS08G. Cross-linked anion exchangers can also used. The preferred degree of crosslinking is about 2% to about 4.5% crosslinking, with a crosslinking of about 40% being particularly preferred.

The adsorbent can be distributed across a number of columns. The number of columns used is one of the parameters that can be used to optimize the process, as is the total volume of adsorbent used. A further parameter that can be adjusted to optimize the process is the number of columns used to achieve specific separations.

Chromatographic purification of sucralose is disclosed by Ratman, U.S. Patent Publication 2007/0270583, the disclosure of which is incorporated herein by reference in its entirety for all purposes. The process is a reverse phase column chromatographic process that uses silanized silica as stationary phase and water or a predominantly aqueous mixture derived from water and small proportion of organic solvents as a mobile phase.

Stream 76 of aqueous mother liquor is fed to chromatography column 78. A stream (80) of water is also fed to column 78. An aqueous impurity-containing stream (82) exits the column and is purged from the process. An aqueous sucralose-containing stream (84) exits the column and can be recycled to first aqueous extract 18. Alternatively or additionally, if, as described below, the first recrystallization is carried out from water, at least a portion of aqueous sucralose-containing stream 84 can be fed to first crystallizer 58.

In one aspect of the invention, when second liquid extractor 22 is present, the stream of first mother liquor 62 is divided. A portion of the first mother liquor is purified by chromatography, and the resulting sucralose-containing stream recycled to the process as described above. Another portion of the first mother liquor is recycled to second liquid extractor 22. The relative amount of first mother liquor 62 sent to each step can be adjusted to prevent the build up of less polar impurities in the overall process. When more of the mother liquor is recycled to second liquid extractor 22, the amount of less polar impurities in the process decreases. However, recycling more liquid to second liquid extractor 22 requires more energy and larger equipment, increasing the cost of operating the process.

Alternatively, in another aspect of the invention, chromatography can be carried out using an organic solvent rather than an aqueous solvent. Catani, U.S. Pat. No. 5,977,349, and Dordick, U.S. Pat. No. 5,128,248, the disclosures of which are incorporated herein by reference in their entirety for all purposes, disclose purification of sucralose using silica gel as the adsorbent and an the organic solvent, such as ethyl acetate or acetone, as the organic solvent. When an organic solvent is used for the chromatography, it is not necessary to replace the organic solvent with water before chromatography. A portion or all of the first mother liquor can be fed directly to a chromatography column. An organic stream containing sucralose and a waste stream are produced. The waste stream is purged from the process, and the organic solvent recovered for reuse. The organic stream containing sucralose can be recycled to at least one of second liquid extractor 22, the fourth liquid extractor 50, and the first crystallizer 58. Alternatively, the organic solvent of the sucralose-containing stream can be replaced with water, as described above, and the resulting aqueous stream recycled to feed stream 10 and/or first aqueous extract 18.

First sucralose product 60 is dissolved in water. The stream of first sucralose product 60 in water is fed to a second crystallizer (86). As described below, recycle streams also can be fed to second crystallizer 86. A second sucralose product and a second mother liquor are produced. Second crystallizer 86 can be any type of crystallizer known in the art, examples of which are listed above. The operating conditions for the crystallizer will depend on the factors listed above. Second sucralose product (88) can be collected by any convenient solid/liquid separation means, examples of which are listed above.

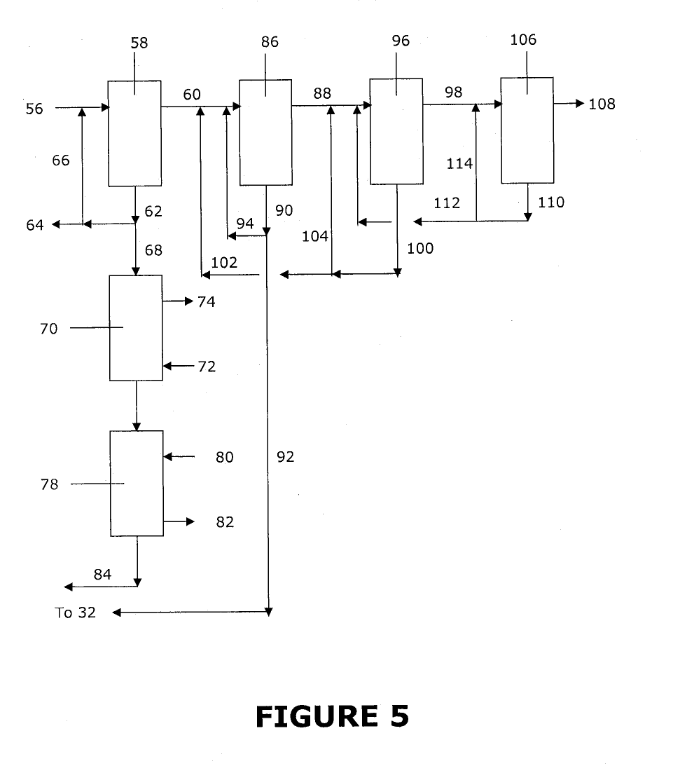

Referring to FIG. 5, second mother liquor (90) can be recycled (stream 92) to first aqueous extract 18, as well as to second crystallizer 86 (stream 94). The fraction of second mother liquor 90 recycled to each process will depend on the product purity and overall yield efficiency desired. If the fraction 92 sent to concentrator 32 is increased, the overall purity of the final product is increased, but plant operation becomes less efficient because larger product streams need to be processed. When the fraction 92 recycled to concentrator 32 is decreased, overall plant productivity, efficiency, and yield are improved, but the product purity is decreased. Alternatively, or additionally, the aqueous solvent in the second mother liquor can be replaced with the second organic solvent, as described above, and the resulting solution recycled to the first crystallizer.

If desired, in one aspect of the invention, second sucralose product 88 can be dissolved in water and sent to third crystallizer 96, where the sucralose is again recrystallized from water under conditions similar to those used in second crystallizer 86. A third sucralose product and a third mother liquor are produced. As described below, recycle streams are also fed to third crystallizer 96. Third sucralose product (98) can be separated from third mother liquor (100) by any convenient solid-liquid separation technique known in the art, examples of which are listed above. Third mother liquor 100 can be recycled to second crystallizer 86 (stream 102) and/or to third crystallizer 96 (stream 104).

If desired, in one aspect of the invention, third sucralose product 98 can be dissolved in water and fed to fourth crystallizer 106, where the sucralose is again recrystallized from water under conditions similar to those used in second crystallizer 86. A fourth sucralose product and a fourth mother liquor are produced. The fourth sucralose product (108) can be separated from the fourth mother liquor (110) by any convenient solid-liquid separation technique known in the art, examples of which are listed above. Fourth mother liquor 110 can be recycled to third crystallizer 96 (stream 112) and/or to fourth crystallizer 106 (stream 114). If desired, the stream of third sucralose product 98 in water can be passed through a column of activated carbon before being fed to fourth crystallizer 106.

A solution of sodium acetate can be added as a buffer to the last crystallizer in the process in which crystallization is carried out from an aqueous solvent, for example: to fourth crystallizer 106, to third crystallizer 96 if fourth crystallizer 106 is not present, or to second crystallizer 86 if third crystallizer 96 and fourth crystallizer 106 are not present. When first crystallizer 50 comprises an aqueous solvent, buffer can also be added to first crystallizer 50 in addition to being added to the other crystallizers. Because of the recycle streams, the buffer is distributed throughout the process. The concentration of sodium acetate is maintained at about 50 to 250 ppm.

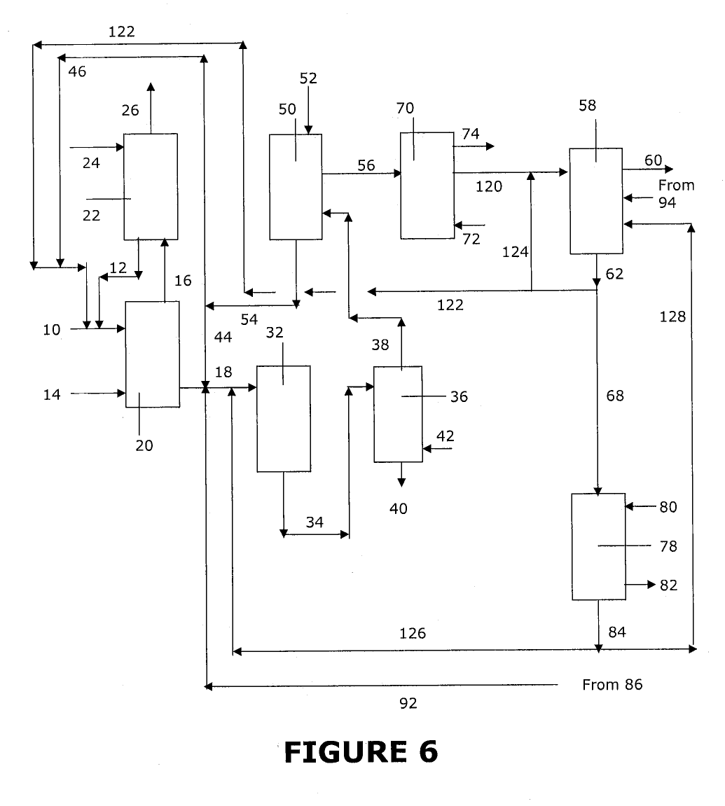

Referring to FIG. 6, in an alternative embodiment of the invention, the organic solvent in fourth organic extract 56 is replaced with water following step EXT2B. This can be conveniently carried out using a distillation column. Fourth organic extract 56 from fourth liquid extractor 50 is fed to a distillation column (70). A stream of water (72) is added and the organic solvent (74) distilled off and recovered for reuse in the process. Alternatively, the organic solvent can be distilled off or stripped off under reduced pressure, and the resulting solid material, the solids formerly dissolved in fourth organic extract 56, dissolved in water.

The resulting aqueous stream, fifth aqueous stream 120, is fed to first crystallizer 58. A first sucralose product and a first mother liquor are produced. A portion (122) of first mother liquor 62 can recycled to first liquid extractor 20. It is also sometimes advantageous to recycle a small stream (124) of the first mother liquor back to first crystallizer 58.

Because the organic solvent has been replaced with water, the first crystallization is carried out from an aqueous solvent instead of from an organic solvent. Because first mother liquor 62 from first crystallizer 58 is an aqueous stream, rather than an organic stream, it is not necessary to replace the organic solvent of first mother liquor 62 with water before the stream is fed to chromatography column 78. A portion (68) of first mother liquor 62 can be fed directly to chromatography column 78. The resulting purified first mother liquor 84 can be fed to first aqueous extract 18 (stream 126) and/or first crystallizer 58 (stream 128). First sucralose product 60 is processed as described above. However, at least a portion of second mother liquor 90 can be recycled to first crystallizer 58.

### Preparation of Sucrose-6-Ester

Selective protection of the 6-hydroxyl of sucrose can be carried out by reaction of sucrose with a carboxylic acid anhydride, such as acetic anhydride or benzoic anhydride, in an anhydrous polar aprotic solvent in the presence of an organotin-based acylation promoter, at a temperature and for a period of time sufficient to produce the sucrose-6-ester. The 6-ester group shields the hydroxyl on the 6 position from the chlorination reaction. Accordingly, any ester group that is stable to the conditions of the chlorination reaction and that can be removed under conditions that do not affect the resulting sucralose can be used. When sucrose-6-acetate is prepared, 1,3-diacetoxy-1,1,3,3-tetrabutyldistannoxane, for example, can be used as the organotin-based acylation promoter and acetic anhydride as the carboxylic acid anhydride. Preparation of sucrose-6-esters is disclosed in, for example, O'Brien, U.S. Pat. No. 4,783,526; Navia, U.S. Pat. No. 4,950,746; Simpson, U.S. Pat. No. 4,889,928; Neiditch, U.S. Pat. No. 5,023,329; Walkup, U.S. Pat. No. 5,089,608; Vernon, U.S. Pat. No. 5,034,551; Sankey, U.S. Pat. No. 5,470,969; Kahn, U.S. Pat. No. 5,440,026; Clark, U.S. Pat. No. 6,939,962, and Li, U.S. Pat. Pub. 2007/0227897 A1; the disclosures of which are all incorporated herein by reference in their entirety for all purposes.

### Preparation of Sucralose-6-Ester Containing Feed Streams

To convert sucrose-6-ester to sucralose-6-ester, the hydroxyls at the 4, 1′, and 6′ positions of the sucrose-6-ester are converted to chloro groups, and the stereochemical configuration at the 4 position is inverted. Conversion of the hydroxyls in the 4, 1′, and 6′ positions of the ester to chloro groups with inversion of the stereochemical configuration at the 4 position is disclosed in Walkup, U.S. Pat. No. 4,980,463; Jai, U.S. Pat. Pub. 2006/0205936 A1; and Fry, U.S. Pat. Pub. 2007/0100139 A1; the disclosures of which are all incorporated herein by reference in their entirety for all purposes.

The chlorination process comprises the following steps. A reaction mixture is prepared comprising the sucrose-6-ester, a tertiary amide, and at least seven molar equivalents of a chlorination agent. For example, in one process, the sucrose-6-ester can be added in a feed stream that comprises about 20 wt % to about 40 wt % of the sucrose-6-ester. The ratio by weight of tertiary amide to total carbohydrate in the reaction mixture is about 5:1 to about 12:1. Alternatively, a preformed chloroformiminium salt, such as (chloromethylene)dimethylammonium chloride (Arnold's reagent), can be used. (Chloromethylene)dimethylammonium chloride can be prepared, for example, by the reaction of phosgene with N,N-dimethyl formamide. Typically, the molar ratio of the (chloromethylene)dimethylammonium salt to the sucrose-6-ester is about 7:1 to about 11:1.

Subsequently, the hydroxyl groups of the sucrose-6-ester at the 2, 3, 4, 1′, 3′, 4′, and 6′ positions are converted to O-alkylformiminium groups. The resulting reaction mixture is heated at a temperature or temperatures and for a period of time or times sufficient to produce a product containing a derivative of sucralose-6-ester in which the remaining hydroxyl groups remain as O-alkylformiminium groups. For example, Walkup, U.S. Pat. No. 4,980,463, the disclosure of which is incorporated herein by reference in its entirety for all purposes, and Fry, U.S. 2007/0100139, the disclosure of which is incorporated herein by reference in its entirety for all purposes, disclose such processes.

Because formation of a chloroformiminium salt or Vilsmeier reagent is not essential to the chlorination reaction, chlorination agent refers to any compound that can be used to form a chloroformiminium salt or Vilsmeier reagent, or that can convert the hydroxyl groups of a sucrose-6-ester to chloro groups. Some chlorination agents that can be used include, for example, phosgene, phosphorus oxychloride, phosphorus pentachloride, thionyl chloride, sulfuryl chloride, oxalyl chloride, trichloromethyl chloroformate (“diphosgene”), bis(trichloromethyl) carbonate (“triphosgene”), and methane sulfonylchloride. Tertiary amides that can be used include, for example, N,N-dimethyl formamide (DMF), N-formyl piperidine, N-formyl morpholine, and N,N-diethyl formamide. When N,N-dimethyl formamide is used as the tertiary amide, it can also be used as the reaction solvent. Cosolvents can be used at up to about 80 vol % or more of the liquid phase of the reaction medium. Useful co-solvents are those which are both chemically inert and which provide sufficient solvent power to enable the reaction to become essentially homogeneous at the monochlorination stage, for example toluene, o-xylene, 1,1,2-trichloroethane, 1,2-diethoxyethane, diethylene glycol dimethyl ether.

Quenching of the reaction mixture restores the hydroxyl groups at the 2, 3, 3′, and 4′ positions and forms the sucralose-6-ester. The reaction mixture can be quenched by the addition of about 0.5 to about 2.0 molar equivalents, typically about 1.0 to about 1.5 molar equivalents, of alkali relative to the amount of chlorination agent used in the reaction. An aqueous solution of an alkali metal hydroxide, such as sodium or potassium hydroxide; an aqueous slurry of an alkaline earth metal hydroxide, such as calcium hydroxide; or aqueous ammonium hydroxide can be used to quench the reaction. For example, an aqueous solution of an alkali metal hydroxide, such as aqueous sodium hydroxide, that contains about 5 wt % to about 35 wt %, typically about 8 wt % to about 20 wt %, and preferably about 10 wt % to about 12 wt % can be used.

As described below, quenching can be carried out by addition of alkali to the reaction mixture, by the dual stream process, or by the circulated process. In each case pH and temperature are controlled during addition of the alkali. Quenching is typically carried out at a pH between about 8.5 to about 10.5 and at a temperature of about 0° C. to about 60° C. Preferably, the pH should not be permitted to rise above about 10.5 during the course of the quenching reaction.

In the dual stream process, quenching is carried out by slow addition of the aqueous alkali with simultaneous slow addition of the chlorination reaction material into a reaction vessel. The chlorination reaction mixture and aqueous alkali are simultaneously added slowly until the desired quantity of chlorination reaction mixture has been added. Further aqueous alkali is added until the desired pH is reached. Then the temperature and pH are maintained at the desired levels for the remainder of the reaction. This process can be a batch or continuous process.

In the circulated process, quenching is carried out by circulating the chlorination reaction mixture from a vessel through a circulation loop. Chlorination reaction mixture and aqueous alkali are added slowly into this circulation loop. Sufficient aqueous alkali is added until the desired pH is reached. Then the temperature and pH are maintained at the desired levels for the remainder of the reaction. This process can be a batch or continuous process.

Following quenching, the reaction mixture can be neutralized by the addition of aqueous acid, for example aqueous hydrochloric acid. The resulting mixture comprises sucralose 6-ester, other carbohydrate including chlorinated carbohydrate impurities, unreacted tertiary amide, and salts in an aqueous solvent in which the predominant solvent is water.

Conversion of Sucralose-6-Ester to Sucralose

The sucralose-6-ester containing aqueous feed stream typically comprises both sucralose and sucralose-6-ester. Methods for hydrolyzing sucralose-6-ester are disclosed, for example in Catani, U.S. Pat. Nos. 5,977,349, 6,943,248, 6,998,480, and 7,049,435; Vernon, U.S. Pat. No. 6,890,581; El Kabbani, U.S. Pat. Nos. 6,809,198, and 6,646,121; Navia, U.S. Pat. Nos. 5,298,611 and 5,498,709, and U.S. Pat. Pub. 2004/0030124; Liesen, U.S. Pat. Pub. 2006/0188629 A1; Fry, U.S. Pat. Pub. 2006/0276639 A1; El Kabbani, U.S. Pat. Pub. 2007/0015916 A1; Deshpande, U.S. Pat. Pub. 2007/0160732 A1; and Ratnam, U.S. Pat. Pub. 2007/0270583 A1; the disclosures of which are all incorporated herein by reference in their entirety for all purposes.

For example, (a) sucralose-6-ester can be hydrolyzed to sucralose by raising the pH of the reaction mixture to about 11±1 at a temperature and for a time sufficient to effect removal of the protecting group, and (b) the tertiary amide is removed by, for example, stream stripping. Either step (a) or step (b) can be carried first. Alternatively, conversion of sucralose-6-ester to sucralose can be carried in methanol containing sodium methoxide. A trans-esterification reaction occurs that forms sucralose and the methyl ester of the acid, for example methyl acetate when the sucralose-6-ester is sucralose-6-acetate. The methyl ester of the acid can be removed by distillation, and the resulting sucralose-containing product dissolved in water.

### INDUSTRIAL APPLICABILITY

The process of the invention is useful in the preparation of sucralose. The invention provides an increased yield of crystalline sucralose from a feed of an impure aqueous sucralose solution such as one obtained by alkaline deacylation of a 6-O-acyl precursor and neutralization.

Sucralose is a high-intensity sweetener that can be used in many food and beverage applications, as well as in other applications. Such applications include, for example, beverages, combination sweeteners, consumer products, sweetener products, tablet cores (Luber, U.S. Pat. No. 6,277,409), pharmaceutical compositions (Luber, U.S. Pat. No. 6,258,381; Roche, U.S. Pat. No. 5,817,340; and McNally, U.S. Pat. No. 5,593,696), rapidly absorbed liquid compositions (Gelotte, U.S. Pat. No. 6,211,246), stable foam compositions (Gowan, Jr., U.S. Pat. No. 6,090,401), dental floss (Ochs, U.S. Pat. No. 6,080,481), rapidly disintegrating pharmaceutical dosage forms (Gowan, Jr., U.S. Pat. No. 5,876,759), beverage concentrates for medicinal purposes (Shah, U.S. Pat. No. 5,674,522), aqueous pharmaceutical suspensions (Ratnaraj, U.S. Pat. No. 5,658,919; Gowan, Jr. U.S. Pat. Nos. 5,621,005 and 5,374,659; and Blase, U.S. Pat. Nos. 5,409,907 and 5,272,137), fruit spreads (Antenucci, U.S. Pat. Nos. 5,397,588; and Sharp, 5,270,071), liquid concentrate compositions (Antenucci, U.S. Pat. No. 5,384,311), and stabilized sorbic acid solutions (Merciadez, U.S. Pat. No. 5,354,902). The determination of an acceptable sweetness can be accomplished by a variety of standard “taste test” protocols known in the art which are well known to those skilled in the art, such as, for example, the protocols referred to in Merkel, U.S. Pat. No. 6,998,144, and Shamil, U.S. Pat. No. 6,265,012.

The advantageous properties of this invention can be observed by reference to the following examples which illustrate but do not limit the invention.

### EXAMPLES

### Example 1

This example was generated using a mathematical model that included both a first extraction process (EXT1), a back extraction (EXT1B) of the first organic extract (16), and recycle of the second aqueous extract (12) to the first extraction process. The calculations used in the model were derived from theoretical equations fitted to actual pilot plant data. FIG. 1 shows a flow diagram of the modeled process.

FIG. 2 shows the results from multiple model runs in which the mass ratio of first organic solvent 14 to the combined aqueous feed stream in the first extraction was varied. The number of separation stages in the back extraction was adjusted to maintain an equivalent overall extraction yield. The amount of sucralose extracted into the first organic extract 16 during the first extraction step is shown on the left hand axis. The purity of the sucralose produced by the process is shown on the right hand axis.

As can be seen from FIG. 2, when the mass ratio of organic solvent 14 to combined aqueous feed stream in the first extraction step is about 0.4 or greater, about 50% or more of the sucralose is extracted into first organic extract 16. When the mass ratio is 0.6 or greater, greater than about 65% of the sucralose is extracted into first organic extract 16. Surprisingly, the level of impurities in first aqueous extract 18 is reduced significantly, with little or no reduction in overall sucralose yield, when higher organic solvent to the combined aqueous feed stream ratios are used in the first extraction step. As can also be seen from FIG. 2, product purity begins to level out near 75%, when nearly 90% of the sucralose is extracted into first organic extract 16.

### Example 2

This example shows the effect of sucralose concentration on the partition coefficient of sucralose between an organic phase and aqueous phase. Aqueous solutions of sucralose were prepared at various carbohydrate concentrations. An equal volume of ethyl acetate was then added to each solution and the two phases mixed thoroughly. After the two phases separated, the carbohydrate concentration in each phase was determined. The K value was calculated by dividing the concentration of sucralose in the ethyl acetate phase by the concentration of sucralose in the aqueous phase. FIG. 3 shows the effect of carbohydrate concentration on the partition coefficient, K. The larger the K value the more readily sucralose is extracted into the ethyl acetate phase.

### Example 3

This example measures the effect of concentration on sucralose yield. Two different organic solvent to sucralose-containing aqueous feed stream ratios were used: about 3.7:1 and about 3.0:1 (volume to volume). The organic solvent was ethyl acetate. The results are shown in Table 1. In Table 1, “Solvent:Feed” is the ratio (volume to volume) of the organic solvent to the sucralose-containing aqueous feed stream. “Carbohydrates” is the wt % of carbohydrates in the sucralose-containing aqueous feed stream. “Salt” is the wt % of salt in the sucralose-containing aqueous feed stream. “Yield” is the percent of sucralose recovered from the organic phase of the extraction. Because multi-stage contacting devices were used for this testing, K values could not be determined directly from this data and compared to the values determined in Example 2.

TABLE 1

| Concentration | Solvent:Feed | Sucralose (wt %) | Salt (%) | Yield (%) |
|---|---|---|---|---|
| Low | 3.68 | 6.97 | 8.37 | 97.54 |
| Low | 2.95 | 6.17 | 7.78 | 97.31 |
| High | 3.69 | 13.77 | 15.00 | 99.99 |
| High | 3.91 | 14.46 | 15.13 | 99.94 |
| High | 3.76 | 14.69 | 14.98 | 99.98 |
| High | 3.18 | 13.82 | 14.95 | 100 |

These experiments show a dramatic increase in extraction efficiency when the sucralose-containing aqueous feed stream is more concentrated. These extraction efficiencies were inserted into a mathematical model of a purification process for sucralose. Increasing the EXT2 extraction efficiency from 97.5% to 99.5% improves the overall yield of sucralose by >5.5%, when using the same solvent to feed ratio and number of extraction stages.

### Example 4

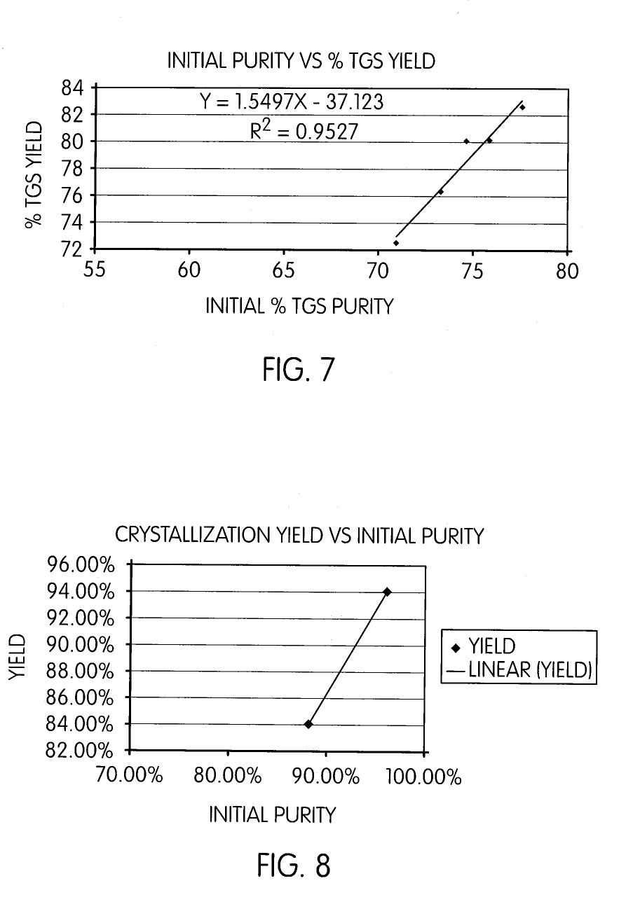

This example shows the influence of feed purity on crystallizer yield. Six different feed solutions of varying feed purity were prepared. Each solution was loaded into a rotary evaporator and heated to a pre-set temperature to make sure all the carbohydrates were completely in solution. Each solution was then cooled to 40° C., and a small amount of sucralose seed crystals were added to each solution. Each solution was then allowed to crystallize for 18 hr. The precipitates were separated from the mother liquor, and a material balance was completed to determine the sucralose yield. The results are shown in FIG. 7. The plot of yield of sucralose (“TGS”) against initial purity has a slope of 1.55, indicating that increasing the feed purity by 2% produces an increase in crystallizer yield of just over 3%.

### Example 5

This example shows the influence of feed purity on crystallizer yield. The procedure of Example 4 was repeated except that two feed solutions of higher purity were used. The results are shown in FIG. 8. This example is similar to Example 4 except that the feed purity for the 2 runs was significantly higher. This experiment indicates that the crystallizer yield increases by a factor of 1.25 for every 1% increase in feed purity.

### Example 6

This example is a mathematical model of the process that determines the influence of solvent to feed ratio and the number of stages in the EXT2B extraction on overall yield of the purification and isolation process. The model is an iterative spreadsheet that links all purification techniques and recycle streams together. The data from Example 4 was used as the basis for the model work presented here.

A base case model was run to determine the purity of the feed to the crystallizer without a back extraction (step EXT2B). Keeping all other parameters constant, two variables were altered to determine the optimal conditions for the extraction: the ratio of water (52) to third organic extract 38 (“EXT2B S:F Ratio”) (volume to volume) and the number of extraction stages (“EXT2B Stages”) in the fourth liquid extraction (step EXT2B). The ratio (“EXT2 S:F Ratio”) of second organic solvent 42 to concentrated feed stream 34 in the third liquid extraction (EXT2) was not varied. “Purity Increase” refers to increase in purity of fourth liquid extract 38. “P1 Increase” refers to the increase in yield in the first crystallization step. “Overall Yield Increase” refers to the yield increase for the entire purification and recovery process. Purity increase was calculated against the base case and this purity multiplied by the crystallization yield factor determined from the data included in Example 4. The projected yield increase was then inserted into the first crystallization part of the spreadsheet and the calculation iterated to steady state. The overall purification area yield produced for each case was then compared against the base case yield to determine the overall yield increase. The results are shown in Table 2.

TABLE 2

| | | EXT2B | EXT2 | | | Overall |
|---|---|---|---|---|---|---|
| | EXT2B | S:F | S:F | Purity | P1 Yield | Yield |
| | Stages | Ratio | Ratio | increase | Increase | Increase |
| Base Case | 0^{a} | 0.00^{a} | 3.0 | 0.00% | 0.00% | 0.00% |
| 1 | 1 | 0.10 | 3.0 | 0.37% | 0.56% | 0.12% |
| 2 | 12 | 0.10 | 3.0 | 0.61% | 0.91% | 0.23% |
| 3 | 12 | 0.30 | 3.0 | 1.36% | 2.04% | 0.52% |
| 4 | 12 | 0.50 | 3.0 | 1.81% | 2.72% | 0.66% |
| 5 | 12 | 0.75 | 3.0 | 2.23% | 3.35% | 0.78% |
| 6 | 6 | 0.75 | 3.0 | 2.20% | 3.30% | 0.77% |
| 7 | 6 | 0.50 | 3.0 | 1.79% | 2.68% | 0.65% |
| 8 | 3 | 0.75 | 3.0 | 2.04% | 3.06% | 0.70% |
| 9 | 1 | 0.75 | 3.0 | 1.36% | 2.04% | 0.44% |
| 10 | 12 | 1.00 | 3.0 | 2.56% | 3.85% | 0.87% |
| 11 | 12 | 1.30 | 3.0 | 2.88% | 4.33% | 0.95% |

^{a}EXT2B step omitted.

The disclosure of the invention includes the following claims. Having described the invention, we now claim the following and their equivalents.

What is claimed is:

1. A process comprising the steps of:
a1) extracting an aqueous feed stream comprising sucralose and chlorinated impurities with a first organic solvent and producing a first organic extract and a first aqueous extract, in which the first organic solvent is immiscible with water, a portion of the sucralose passes into the first organic extract and a portion of the sucralose is retained in the first aqueous extract, and a portion of the chlorinated impurities passes into the first organic extract; and
a2) extracting the first organic extract with an aqueous solvent and producing a second organic extract and a second aqueous extract, and adding the second aqueous extract to step a1), in which the sucralose preferentially passes into the second aqueous extract, and at least a portion of the chlorinated impurities is retained in the second organic extract;
b) optionally, concentrating the first aqueous extract;
c1) extracting the first aqueous extract with a second organic solvent to give a third aqueous extract and a third organic extract, in which the second organic solvent is immiscible with water, the sucralose preferentially passes into the third organic extract, and a portion of the chlorinated impurities are retained in the third aqueous extract;
c2) optionally, extracting the third organic extract with water to produce a fourth organic extract and a fourth aqueous extract, in which at least a portion of the sucralose is retained in the fourth organic extract, and adding the fourth aqueous extract to at least one of the first aqueous extract and the aqueous feed stream;
d) crystallizing the sucralose from the organic extract produced in step c) and producing a first sucralose product and a first mother liquor, the first mother liquor comprising sucralose and a portion of the chlorinated impurities;
e) carrying out step e1) and optionally step e2):
e1) adding at least a portion of the first mother liquor to step a2);
e2a) optionally, replacing the second organic solvent of at least a portion of the first mother liquor with water; and
e2b) chromatographing the first mother liquor and producing a purified first mother liquor and a waste stream, in which the sucralose preferentially passes into the purified first mother liquor and at least a portion of the chlorinated impurities preferentially pass into the waste stream; and adding at least a portion of the purified first mother liquor either (i) to at least one of the aqueous feed stream and the first aqueous extract, or (ii) to at least one of step a2), step c2), and step d); and
f) recrystallizing the first sucralose product from an aqueous solvent and producing a second sucralose product and a second mother liquor.

2. The process of claim 1 in which step b) is carried out.

3. The process of claim 1 in which step c2) is carried out.

4. The process of claim 1 additionally comprising a step of extracting the third aqueous extract with the first organic solvent or the second organic solvent to obtain a resulting organic extract and adding the resulting organic extract to at least one of step a2) and step c1).

5. The process of claim 1 in which step e2a) is carried out.

6. The process of claim 1 in which both step e2a) and step e2b) are carried out.

7. The process of claim 1 in which step e2b) is carried out and at least a portion of the purified first mother liquor is added to at least one of the first aqueous extract, the aqueous feed stream, step a2), step c2), or step d).

8. The process of claim 1 additionally comprising a step of adding at least a portion of the second mother liquor to the first aqueous extract.

9. The process of claim 1 additionally comprising a step of adding at least a portion of the second mother liquor to step f).

10. The process of claim 1 additionally comprising a step of replacing the water of at least a portion of the second mother liquor with the second organic solvent and adding at least a portion of the resulting second mother liquor containing the second organic solvent to step d).

11. The process of claim 1 additionally comprising a step of:
g) recrystallizing the second sucralose product from an aqueous solvent and forming a third sucralose product and a third mother liquor.

12. The process of claim 1 wherein a buffer is added to at least one of step f), step g), and step h), in which: the buffer is added to step h) when step g) and step h) are present, the buffer is added to step g) when step g) is present and step h) is absent, and the buffer is added to step f) when both step g) and step h) are absent, wherein step g) comprises recrystallizing the second sucralose product from an aqueous solvent and forming a third sucralose product and a third mother liquor and step h) comprises recrystallizing the third sucralose product from an aqueous solvent and forming a fourth sucralose product and a fourth mother liquor.

13. A process comprising the steps of:
a1) extracting an aqueous feed stream comprising sucralose and chlorinated impurities with a first organic solvent and producing a first organic extract and a first aqueous extract, in which the first organic solvent is immiscible with water, a portion of the sucralose passes into the first organic extract and a portion of the sucralose is retained in the first aqueous extract, and a portion of the chlorinated impurities passes into the first organic extract; and
a2) optionally, extracting the first organic extract with an aqueous solvent and producing a second organic extract and a second aqueous extract, and adding the second aqueous extract to step a1), in which the sucralose preferentially passes into the second aqueous extract, and at least a portion of the chlorinated impurities is retained in the second organic extract;
b) optionally, concentrating the first aqueous extract;
c1) extracting the first aqueous extract with a second organic solvent to give a third aqueous extract and a third organic extract, in which the second organic solvent is immiscible with water, the sucralose preferentially passes into the third organic extract, and a portion of the chlorinated impurities are retained in the third aqueous extract;
c2) optionally, extracting the third organic extract with water to produce a fourth organic extract and a fourth aqueous extract, in which at least a portion of the sucralose is retained in the fourth organic extract, and adding the fourth aqueous extract to at least one of the first aqueous extract and the aqueous feed stream;
d) replacing the second organic solvent of the organic extract produced in step c) with water and producing a fifth aqueous extract;
e) crystallizing the sucralose from the fifth aqueous extract and producing a first sucralose product and a first mother liquor, the first mother liquor comprising sucralose and chlorinated impurities;
f) carrying out at least one of steps f1) and f2):
f1) adding at least a portion of the first mother liquor to step a1);
f2) chromatographing at least a portion of the first mother liquor and producing a purified first mother liquor and a waste stream, in which the sucralose preferentially passes into the purified first mother liquor and chlorinated impurities preferentially pass into the waste stream, and adding at least a portion of the purified first mother liquor to at least one of the first aqueous extract and step e);
g) recrystallizing the first sucralose product from an aqueous solvent and producing a second sucralose product and a second mother liquor.

14. The process of claim 13 in which step b) is carried out.

15. The process of claim 13 in which step c2) is carried out.

16. The process of claim 13 additionally comprising a step of extracting the third aqueous extract with the first organic solvent or the second organic solvent to obtain a resulting organic extract and adding the resulting organic extract to at least one of step a2) and step c1).

17. The process of claim 13 in which step a2) is carried out.

18. The process of claim 13 in which step f1) is carried out.

19. The process of claim 13 in which step f2) is carried out.

20. The process of claim 13 in which both step f1) and step f2) are carried out.

21. The process of claim 13 additionally comprising adding at least a portion of the first mother liquor to step e).

22. The process of claim 13 in which step f2) is carried out and at least a portion of the purified first mother liquor is added to at least one of the first aqueous extract, the aqueous feed stream, step a2), or step c2).

23. The process of claim 13 additionally comprising a step of adding at least a portion of the second mother liquor to step e).

24. The process of claim 13 additionally comprising a step of adding at least a portion of the second mother liquor to step g).

25. The process of claim 13 additionally comprising a step of adding at least a portion of the second mother liquor to the first aqueous extract.

26. The process of claim 13 additionally comprising a step of:
h) recrystallizing the second sucralose product from an aqueous solvent and forming a third sucralose product and a third mother liquor.

27. The process of claim 13 wherein a buffer is added to one of step g), step h), and step i), and, optionally, to step e), in which: the buffer is added to step i) when step i) and step h) are present, the buffer is added to step h) when step h) is present and step i) is absent, and the buffer is added to step g) when both step h) and step i) are absent, wherein step h) comprises recrystallizing the second sucralose product from an aqueous solvent and forming a third sucralose product and a third mother liquor and step i) comprises recrystallizing the third sucralose product from an aqueous solvent and forming a fourth sucralose product and a fourth mother liquor.
